(19) United States
(12) Patent Application Publication
(10) Pub. No.: US 2025/0100982 A1
(43) Pub. Date: Mar. 27, 2025

(54) PLEUROMUTILIN DERIVATIVE WITH THIAZOLE SIDE CHAIN AND PREPARATION AND APPLICATION

(71) Applicant: SOUTH CHINA AGRICULTURAL UNIVERSITY, Guangzhou (CN)

(72) Inventors: Youzhi TANG, Guangzhou (CN); Ke LI, Guangzhou (CN); Zhen JIN, Guangzhou (CN); Zhenling ZENG, Guangzhou (CN); Xianhui HUANG, Guangzhou (CN); Huanzhong DING, Guangzhou (CN)

(21) Appl. No.: 18/475,173

(22) Filed: Sep. 26, 2023

(51) Int. Cl. C07D 277/54; A61P 31/04

(52) U.S. Cl. CPC C07D 277/54; A61P 31/04

(57) ABSTRACT

The present invention belongs to the field of medicinal chemistry, and particularly relates to a pleuromutilin derivative with a thiazole side chain and preparation and application. The pleuromutilin derivative with the thiazole side chain is a compound of formula 2 or a pharmaceutically acceptable salt thereof, and a solvent compound, enantiomer, diastereoisomer, tautomer, or mixture of any proportion of the compound of formula 2 or the pharmaceutically acceptable salt thereof, comprising racemic mixtures: The pleuromutilin derivative has good antibacterial activity, is particularly suitable for use as a novel antibacterial drug for animal or human systemic infection, and has good water solubility.

### TECHNICAL FIELD

The present invention belongs to the field of medicinal chemistry, in particular to a pleuromutilin derivative with a thiazole side chain and preparation and application.

### BACKGROUND OF THE PRESENT INVENTION

With the abuse of antibiotics, the bacterial drug resistance has become more and more serious. Wherein methicillin-resistant Staphylococcus aureus (MRSA) is a typical representative of drug-resistant bacteria. Multidrug-resistant Staphylococcus aureus (MRSA) is an important zoonotic pathogen, which may cause infections of skin, soft tissue, etc. MRSA infections are reported in the world and continue to increase in prevalence in communities and hospitals, thereby posing a serious threat to the human health of the world. Therefore, there is an urgent need to develop anti-MRSA drugs with unique antimicrobial mechanisms to deal with the potential harms caused by the MRSA infections.

Pleuromutilin (formula 1) is a natural diterpenoid compound with a tricyclic skeleton, which was isolated and purified from the cultures of Pleurotus mutilu and Pleurotus passeckerianus by Kavanagh et al. in the 1950s.

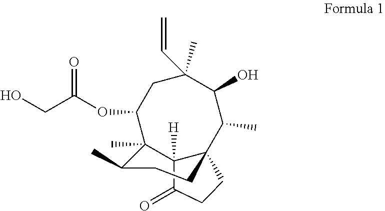 [SMILES: C=C[C@]1(C)C[C@@H](OC(=O)CO)[C@]2(C)[C@H](C)CC[C@]3(CCC(=O)[C@H]32)[C@@H](C)[C@@H]1O]

Studies have shown that the pleuromutilin has strong antibacterial activity against most Gram-positive bacteria and mycoplasma, and mainly interacts with the V region on the 50S subunit of bacterial ribosomes to affect the protein synthesis of bacteria to perform the effect. Due to the unique mechanism of action, the pleuromutilin and its derivatives are not easy to produce cross resistance with other drugs. At the same time, the researchers have found that the C14 side chain group on the structural molecule of the pleuromutilin can be changed to enhance the interaction with peptidyl transferase, so as to design the derivatives with excellent antibacterial activity.

At present, there are 4 kinds of pleuromutilin drugs on the market at home and abroad, among which tiamulin and valnemulin are two special antimicrobial drugs for animals. Retapamulin, approved by the U.S. Food and Drug Administration (FDA) in 2007, is the first pleuromutilin antibacterial drug for human use. Lefamulin is the first oral and intravenous pleuromutilin antibacterial drug for human use.

Compared with the drugs successfully developed based on the same parent nucleus, such as dozens of antibacterial drugs of penicillin, cephalosporin, and floxacin, only four kinds of antibacterial drugs of the pleuromutilin are successfully developed, and drug-resistant bacteria against the antibacterial drugs of the pleuromutilin are still rare. Therefore, it is necessary to develop more antibacterial drugs of the pleuromutilin.

### SUMMARY OF PRESENT INVENTION

In order to overcome the shortcomings and disadvantages of the prior art, the main purpose of the present invention is to provide a pleuromutilin derivative with a thiazole side chain. This pleuromutilin derivative has good antibacterial activity and is particularly suitable for use as a novel antibacterial drug for animal or human systemic infection.

Another purpose of the present invention is to provide a preparation method for the pleuromutilin derivative with the thiazole side chain.

Another purpose of the present invention is to provide application of the pleuromutilin derivative with the thiazole side chain.

Another purpose of the present invention is to provide the uses of the pleuromutilin derivative with the thiazole side chain in the preparation of drugs for the treatment of infectious diseases, particularly infectious diseases caused by drug-resistant Staphylococcus aureus, or multidrug-resistant bacteria, or mycoplasma.

The purposes of the present invention are achieved by the following technical solution:

A pleuromutilin derivative with a thiazole side chain is a compound of formula 2 or a pharmaceutically acceptable salt thereof, and a solvent compound, enantiomer, diastereoisomer, tautomer, or mixture of any proportion of the compound of formula 2 or the pharmaceutically acceptable salt thereof, comprising racemic mixtures:

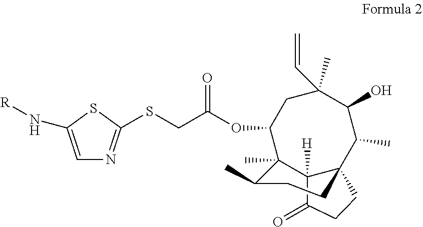

wherein R is cyclopropyl, cyclobutyl, cyclopentyl, cyclohexyl,

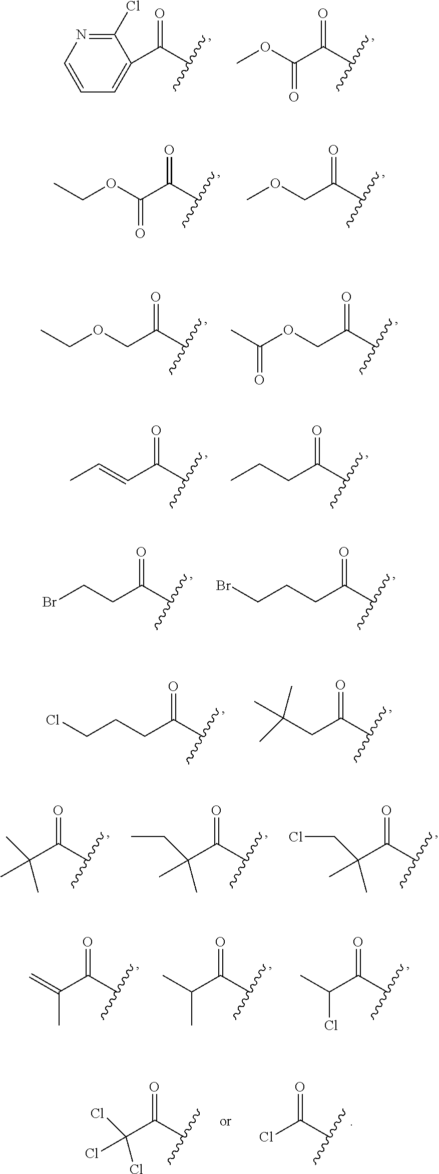

The specific groups of the compounds with the above preferred structure are summarized as shown in Table 1:

Compound numbers and specific groups

| Compound | R |
|---|---|
| 1 | 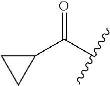 [SMILES: *C(=O)C1CC1] |
| 2 | 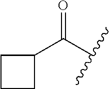 [SMILES: *C(=O)C1CCC1] |
| 3 | 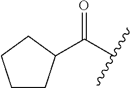 [SMILES: *C(=O)C1CCCC1] |
| 4 | 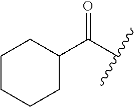 [SMILES: *C(=O)C1CCCCC1] |
| 5 | 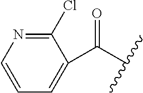 [SMILES: *C(=O)c1cccnc1Cl] |
| 6 | 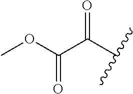 [SMILES: *C(=O)C(=O)OC] |
| 7 | 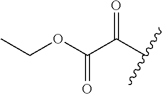 [SMILES: *C(=O)C(=O)OCC] |
| 8 | 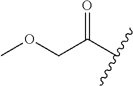 [SMILES: *C(=O)COC] |
| 9 | 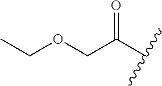 [SMILES: *C(=O)COCC] |
| 10 | 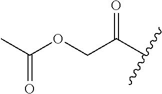 [SMILES: *C(=O)COC(C)=O] |
| 11 | 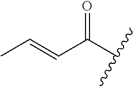 [SMILES: *C(=O)/C=C/C] |
| 12 | 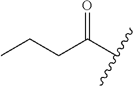 [SMILES: *C(=O)CCC] |
| 13 | 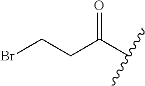 [SMILES: *C(=O)CCBr] |
| 14 | 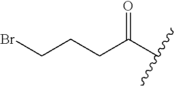 [SMILES: *C(=O)CCCBr] |
| 15 | 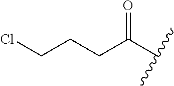 [SMILES: *C(=O)CCCCl] |
| 16 | 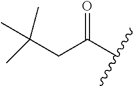 [SMILES: *C(=O)CC(C)(C)C] |
| 17 | 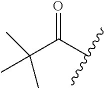 [SMILES: *C(=O)C(C)(C)C] |
| 18 | 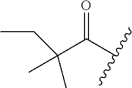 [SMILES: *C(=O)C(C)(C)CC] |
| 19 | 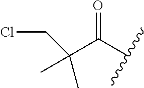 [SMILES: *C(=O)C(C)(C)CCl] |
| 20 | 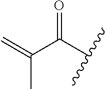 [SMILES: *C(=O)C(=C)C] |
| 21 | 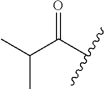 [SMILES: *C(=O)C(C)C] |
| 22 | 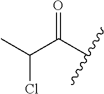 [SMILES: *C(=O)C(C)Cl] |
| 23 | 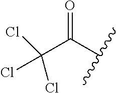 [SMILES: *C(=O)C(Cl)(Cl)Cl] |
| 24 | 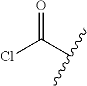 [SMILES: *C(=O)Cl] |

The pharmaceutically acceptable salt is a salt formed by the compound of formula 2 with hydrochloric acid, hydrobromic acid, sulfuric acid, nitric acid, phosphoric acid, acetic acid, fumaric acid, maleic acid, oxalic acid, malonic acid, succinic acid, citric acid, malic acid, methanesulfonic acid, ethanesulfonic acid, benzenesulfonic acid, toluenesulfonic acid, glutamic acid or aspartic acid.

The pharmaceutically acceptable salt preferably has the following structural formula:

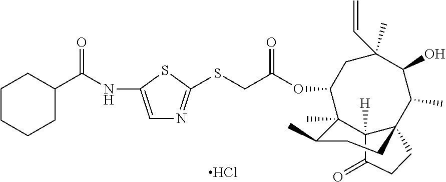 [SMILES: C=C[C@]1(C)C[C@@H](OC(=O)CSc2ncc(NC(=O)C3CCCCC3)s2)[C@]2(C)[C@H](C)CC[C@]3(CCC(=O)[C@H]32)[C@@H](C)[C@@H]1O ; Cl]

The preparation method for the pleuromutilin derivative with the thiazole side chain comprises the following steps:
(1) making pleuromutilin react with p-toluenesulfonyl chloride to obtain an intermediate I with a structure shown in formula 3;
(2) making the intermediate I prepared in step (1) react with sodium iodide to obtain an intermediate II with a structure shown in formula 4;
(3) making aminoacetonitrile hydrochloride as raw material react with CS2 to obtain an intermediate III with a structure shown in formula 5;
(4) making the intermediate II prepared in step (2) react with the intermediate III prepared in step (3) to obtain an intermediate IV with a structure shown in formula 6;
(5) making the intermediate IV prepared in step (4) react with various acyl chlorides to obtain the pleuromutilin derivative with the thiazole side chain with a structure shown in formula 2.

The intermediates I, II, III and IV have structural formulas 3 to 6 respectively:

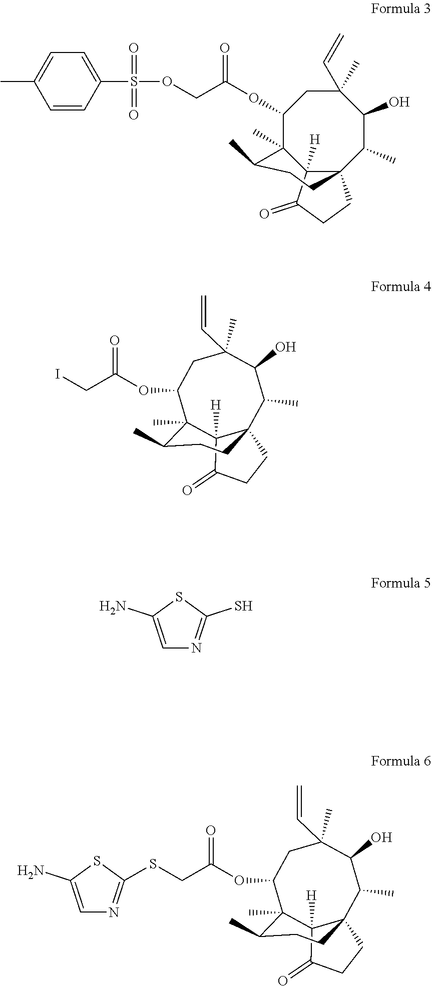

The molar ratio of the p-toluenesulfonyl chloride to the pleuromutilin in step (1) is preferably 1.1:1.

The reaction in step (2) preferably uses anhydrous methanol as a solvent; the molar ratio of the intermediate I to sodium iodide is preferably 1:1, and the reaction condition is preferably 75° C. for 2-3 h.

The molar ratio of the aminoacetonitrile hydrochloride to CS2 in step (3) is preferably 1:1.1.

The specific operation of the reaction in step (4) preferably comprises:
adding the intermediate II to the intermediate III in an ice bath, and then adding sodium methanol, wherein the molar ratio of the sodium methanol to the intermediate II is preferably 1:1.

The specific operation of the reaction in step (5) preferably comprises:
dropwise adding acyl chloride to the intermediate IV at normal temperature, with the molar ratio of 1:1.1 of the intermediate IV to the acyl chloride preferably, and dropping a few drops of triethylamine into the reaction solution as a catalyst, with reaction time of 2-4 h.

A synthetic route is shown as follows:

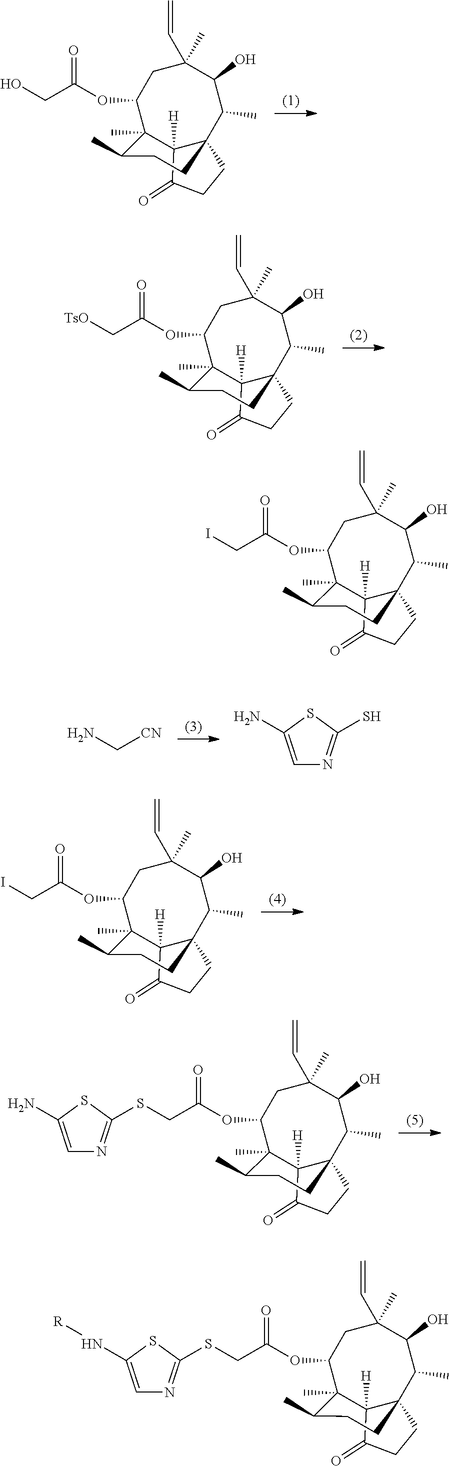

The application of the pleuromutilin derivative with the thiazole side chain in the preparation of antibacterial products is provided.

The antibacterial products are preferably drugs for the treatment of infectious diseases.

The antibacterial products are further preferably antibacterial drugs for treating infectious diseases caused by gram-positive bacteria.

The infectious diseases are caused by infection of drug-resistant Staphylococcus aureus or multi-drug resistant bacteria in humans or animals.

The drugs may contain one or more pharmaceutically acceptable carriers, excipients or diluents.

The formulations of the drugs comprise many clinical drug dosage forms, such as tablets, injections, liposome nanoparticles, controlled release formulations, etc.

An antibiotic drug contains an effective quantity of pleuromutilin derivative with the thiazole side chain, and a residual quantity of pharmaceutic adjuvants or other compatible drugs.

The pharmaceutic adjuvants refer to conventional pharmaceutical excipients, such as solvents, disintegrants, corrigents, preservatives, colorants and adhesives.

The other compatible drugs refer to an effective dose of pleuromutilin derivative with the thiazole side chain as drug raw material compounded with other natural drugs or chemicals.

Compared with the prior art, the present invention has the following advantages and beneficial effects:
(1) The pleuromutilin derivative provided by the present invention is a new type of compound that has not been reported before.
(2) Through extensive and in-depth research, the present invention synthesizes a large number of pleuromutilin derivatives with the thiazole side chains with new structures, and carries out extensive antibacterial activity screening. The present invention finds for the first time that such compounds not only have good in vitro antibacterial activity, but also have the advantage of lower preparation cost compared with valnemulin and retapamulin, and thus are particularly suitable for use as a novel antibacterial drug for the prevention and treatment of human or animal bacterial infectious diseases, especially the infectious diseases caused by drug-resistant Staphylococcus aureus.
(3) The pleuromutilin derivative with the thiazole side chain prepared by the present invention has good water solubility.

### DESCRIPTION OF THE DRAWINGS

FIG. 1 is a nuclear magnetic map of compound 2.

FIG. 2 is a nuclear magnetic map of compound 4.

FIG. 3 is a nuclear magnetic map of compound 10.

FIG. 4 is a nuclear magnetic map of compound 18.

FIG. 5 is a nuclear magnetic map of compound 23.

FIG. 6 shows the toxicity experiment results of some compounds.

### DETAILED DESCRIPTION OF PREFERRED EMBODIMENTS

The present invention is further described in detail below in combination with embodiments and drawings, but the implementation modes of the present invention are not limited herein.

In the embodiments, the specific R groups in thiazole are all commercially available, and other reagents are also commercially available.

### Embodiment 1

(1) Preparation of intermediate I: 10.0 g (26.5 mmol) of pleuromutilin was dissolved in 20 ml of pyridine and placed in an ice bath; 5.6 g (29.2 mmol) of p-toluenesulfonyl chloride was dissolved in 10 ml of pyridine; then the above pleuromutilin pyridine solution was slowly added; after the mixed solution was stirred in the ice bath for 3 h, 50 ml of ice water and 50 ml of trichloromethane were added successively, and then the solution was transferred to a separatory funnel, shaken and stood for stratification; the organic phase was taken and washed with 100 ml of sulfuric acid (4 mol/L), 100 ml of saturated sodium bicarbonate solution and 100 ml of deionized water successively; after washing, the organic solution evaporated under reduced pressure from the organic phase, and 20 ml of isopropyl alcohol was added to the remaining solid; after heated and dissolved, the solution was cooled; a large amount of white powder was precipitated, pumped and filtered; and the filter residue was washed with isopropyl alcohol and dried to obtain the intermediate I with the structure shown in formula 3, with a yield of 81%.
(2) Preparation of intermediate II: 11.7 g of intermediate I (80 mmol) and 3.29 g of sodium methanol (80 mmol) were added to anhydrous methanol and stirred at 75° C. for 2 hours to obtain the intermediate II with the structure shown in formula 4, with a yield of 84.21%.
(3) Preparation of intermediate III: 10 g of aminoacetonitrile (108 mmol) was dissolved in anhydrous methanol, and 9.05 g of carbon disulfide (118 mmol) was slowly dropped to the reaction solution to react under an ice salt bath for 1 hour; and a large amount of solid was precipitated, pumped and filtered to obtain yellow solid intermediate III, with a yield of 45.17%.
(4) Preparation of intermediate IV: 11.7 g of intermediate III (80 mmol) and 11.9 g of sodium methoxide (80 mmol) were added to the intermediate II (80 mmol) to react under the ice salt bath for 2 hours; then, the reaction solution was rotated and dried by distillation; the reactants were redissolved with dichloromethane; the reaction solution was poured into the separatory funnel, and 50 ml of distilled water and 50 ml of dichloromethane were added successively; after shaking, the solution was stood for stratification; and the organic phase was washed twice with distilled water and dried with anhydrous sodium sulfate to obtain the organic phase. The obtained organic phase was rotted and evaporated to obtain a mixture, and the mixture was redissolved by dichloromethane and fully mixed with 10 g of silica gel with 100-200 meshes. After the solvent volatilized, the above crude product-silica gel powder mixture was purified by column chromatography (the silica gel powder with 100-200 meshes was a stationary phase, and dichloromethane: methanol=200:1 (V:V) is a mobile phase), to obtain the intermediate IV, with a yield of 45.65%.

### Embodiment 2

### Preparation of 22-((5-(cyclopropylaminothiazol-2-yl)-sulfur)-deoxypleuromutilin (compound 1)

1 g of intermediate IV (2 mmol) was dissolved in dichloromethane, and cyclopropanecarboxylic acid chloride (2.2 mmol) was dropped into the reactant to react at room temperature for 3 hours. The reaction solution was poured into the separatory funnel; 50 ml of distilled water and 50 ml of dichloromethane were added successively; then the solution was shaken and stood for stratification; the organic phase was taken, washed with distilled water twice, and dried with anhydrous sodium sulfate to obtain the organic phase. The obtained organic phase was rotated and evaporated to obtain a mixture, and the mixture was redissolved by dichloromethane and fully mixed with 1 g of silica gel with 100-200 meshes. After the solvent volatilized, the above crude product-silica gel powder mixture was purified by column chromatography (the silica gel powder with 100-200 meshes was a stationary phase, and dichloromethane: methanol=60:1 (V:V) is a mobile phase), to obtain the compound 1 with the structure shown in formula 6, with a yield of 48.0%.

### Embodiment 3

### Preparation of Compounds 2-24

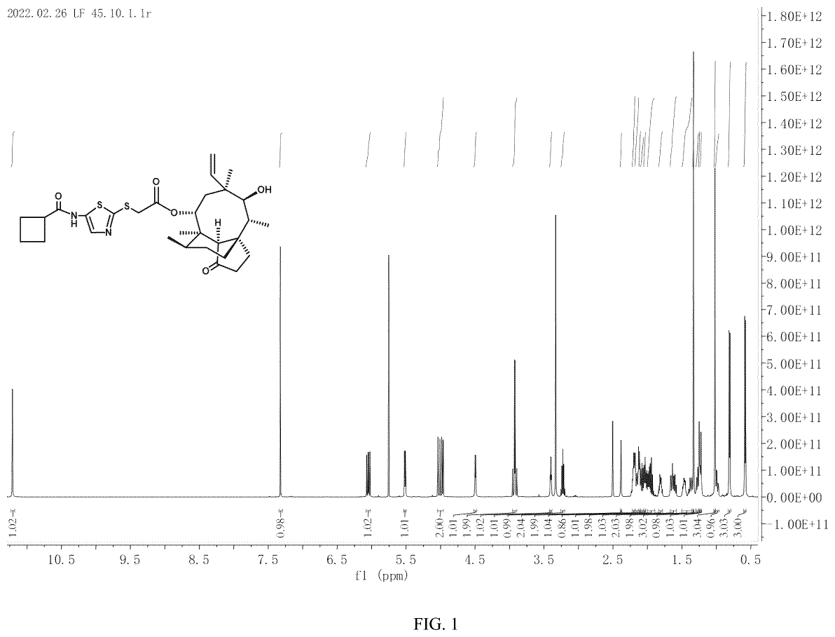

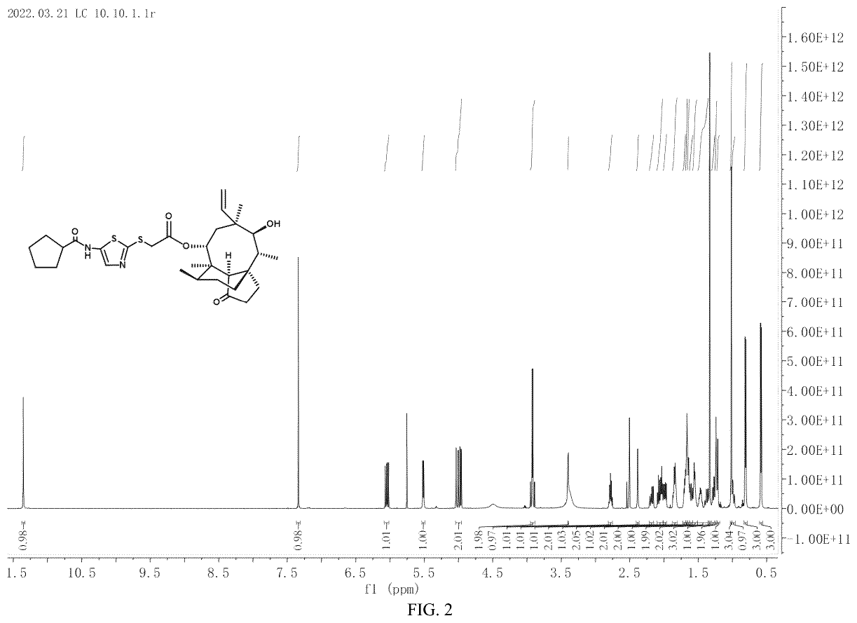

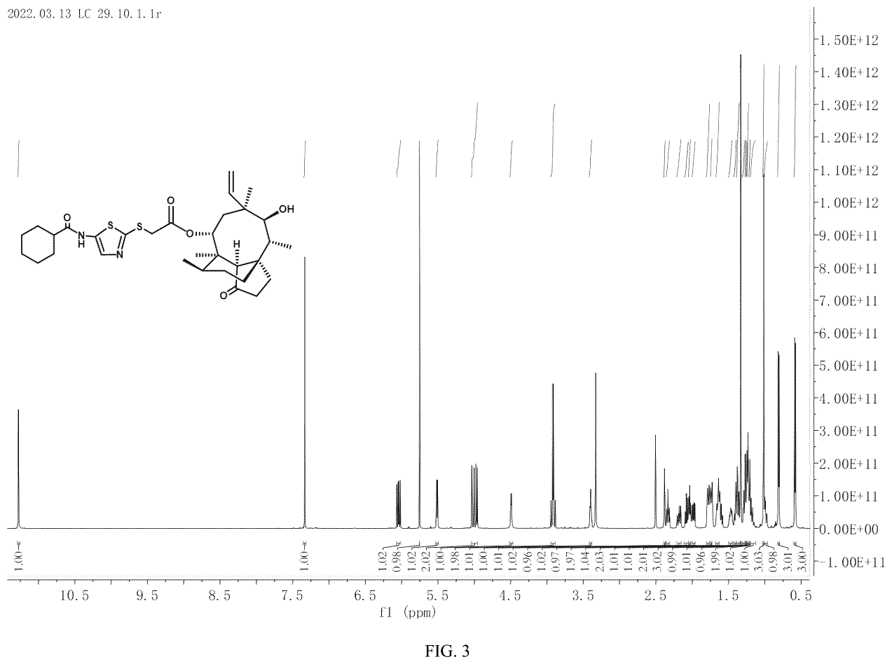

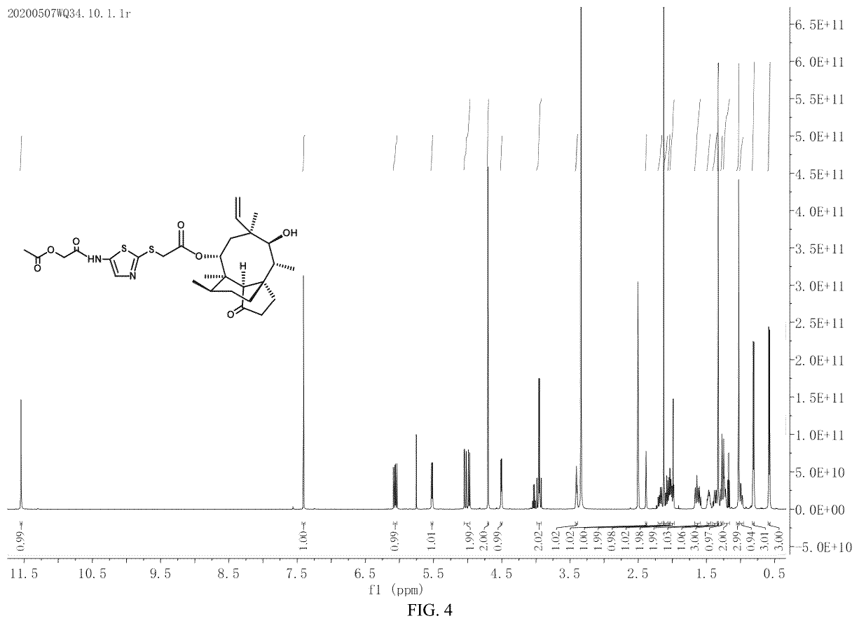

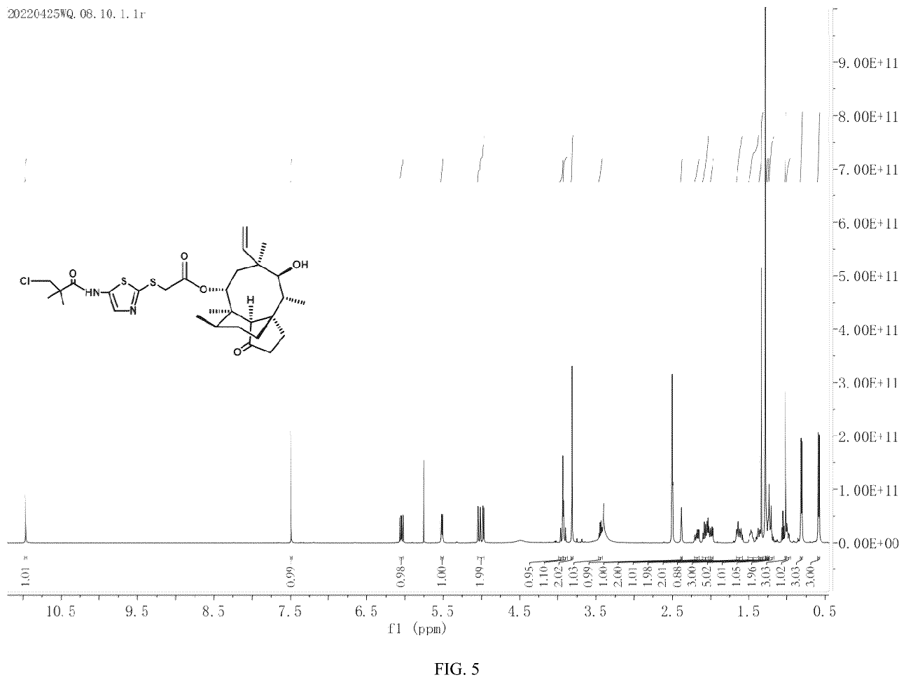

According to the same method of embodiment 2 (the molar quantity of each reactant, reaction conditions, purification, etc. are the same as those of embodiment 2), the corresponding products shown in formula 2 are obtained, and numbered as 2 to 24 in sequence. Wherein FIGS. 1 to 6 are the nuclear magnetic maps of compounds 2, 4, 10, 18 and 23.

The preparation yields of the above compounds are summarized as shown in Table 2.

TABLE 2
Compound numbers and yields

| Compound | Yield (%) |
|---|---|
| 1 | 46.1 |
| 2 | 48.0 |
| 3 | 50.0 |
| 4 | 45.1 |
| 5 | 17.2 |
| 6 | 44.0 |
| 7 | 40.7 |
| 8 | 26.6 |
| 9 | 46.8 |
| 10 | 42.4 |
| 11 | 16.4 |
| 12 | 20.5 |
| 13 | 35.4 |
| 14 | 30.0 |
| 15 | 58.3 |
| 16 | 11.8 |
| 17 | 20.4 |
| 18 | 15.7 |
| 19 | 20.7 |
| 20 | 36.5 |
| 21 | 30.7 |
| 22 | 50.3 |
| 23 | 23.3 |
| 24 | 50.4 |

### Effect Embodiment

(1) In vitro antibacterial experiment

A broth dilution method was used in the experiment. Tiamulin was selected as a control drug in the experiment. The tiamulin is a pleuromutilin antibiotic as well as one of the top ten veterinary antibiotics in the world.

The strains used in the experiment were methicillin-resistant Staphylococcus aureus ATCC43300, Staphylococcus aureus ATCC29213, clinical Staphylococcus aureus AD3, clinical S. aureus 144, Mycoplasma gallisepticum 352, 352 tiamulin induced drug-resistant bacteria and clinical isolates M47.

Preparation of target compound stock solution: 6.4 mg of target compound was accurately weighed, placed in a 5 mL volumetric flask, and dissolved with 0.25 mL of DMSO and 0.25 mL of Tween 80; then 4.5 mL of distilled water was added for fixing the volume; the solution was fully shaken to obtain a stock solution (1280 μg/mL); and the stock solution was filtered with a 0.22 μm filter membrane and sterilized, subpackaged into small tubes, and stored at −20° C. The control drug tiamulin was also prepared according to the above method.

Preparation of bacterial solution: the strains that were well preserved at −20° C. were taken out and inoculated on a new MH plate, and after culture at 37° C. for 24 h, a single colony was selected and inoculated in MH medium for culturing again for 24 h. The selected single colony was transferred to sterile normal saline and the turbidity was adjusted to 0.6 McF. At this moment, the concentration of the bacterial solution was 10⁶ CFU/mL.

Preparation of MIC plate: a sterile 96-well plate was taken; 180 μL of MH broth medium was added to the first well, 100 μL of MH broth medium was added to the second well to the 12^{th} well; 20 μL of target compound solution with a concentration of 1280 μg/mL was added to the first well; after even mixing, 100 μL of solution was added to the second well, and mixed well; 100 μL of solution was absorbed to the third well, and so on; and 100 μL of solution was absorbed to the 12^{th} well and discarded. At this moment, the drug concentrations in the wells were: 128, 64, 32, 16, 8, 4, 2, 1, 0.5, 0.25, 0.125, and 0.06 μg/mL, and three parallel groups were made for the drugs of the concentrations.

Inoculation of bacterial solution: 100 μL of bacterial solution was added into the first well to the 12^{th} well, so that the final bacterial solution concentration in each well was about 5×10⁵ CFU/mL, and the drug concentrations in the first well to the 12^{th} well were 64, 32, 16, 8, 4, 2, 1, 0.5, 0.25, 0.125, 0.06 and 0.03 μg/mL respectively. The inoculated 96-well plate was cultured in an incubator at 37° C., and the growth of the bacterial solution was observed for 24 h. The control drug tiamulin was measured with the same method to completely inhibit the minimum drug concentration of bacterial growth as MIC in the small wells, and the bacteria in the positive control wells (without drug) needed to grow obviously. When a single jumping hole occurred in the microbroth dilution method, the maximum drug concentration for inhibiting bacteria was recorded, and the experiment shall be repeated if multiple jumping holes occurred.

The MIC results of the target compounds against the tested strains are shown in Table 3.

TABLE 3
Results of drug sensitivity of some pleuromutilin drugs to four drug-
resistant Staphylococcus aurei

| | | MRSA | | | |
|---|---|---|---|---|---|
| | | ATCC | S. aureus | S. aureus | S. aureus |
| Compound | R = | 43300 | ATCC 29213 | AD3 | 144 |
| 2 | 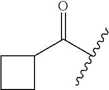 [SMILES: *C(=O)C1CCC1] | 0.0625 | 0.125 | 0.125 | 0.25 |
| 4 | 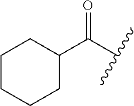 [SMILES: *C(=O)C1CCCCC1] | 0.125 | 0.03125 | 0.0625 | 0.0625 |
| 10 | 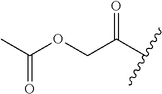 [SMILES: *C(=O)COC(C)=O] | 0.0625 | 0.125 | 0.125 | 0.0625 |
| 18 | 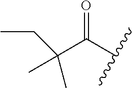 [SMILES: *C(=O)C(C)(C)CC] | 0.25 | 0.25 | 0.25 | 0.5 |
| 23 | 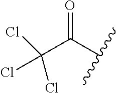 [SMILES: *C(=O)C(Cl)(Cl)Cl] | 1 | 2 | 1 | 2 |
| Tiamulin | | 0.5 | 1 | 1 | 1 |

(2) In vitro inhibition experiment of mycoplasma activity

The broth dilution method was adopted in the experiment. The tiamulin was selected as the control drug in the experiment. The tiamulin is a pleuromutilin antibiotic and mainly used to prevent mycoplasma hyopneumoniae, etc., and has a better effect on mycoplasma than macrolide.

The strains used in the experiment were Mycoplasma gallisepticum 352, 352-tiamulin resistant strain, and M47.

Preparation of the target compound stock solution: the compound was dissolved with 1 mL of DMSO, then fixed in volume with a 250 mL volumetric flask and fully shaken to obtain the stock solution (5120 μg/mL).

The stock solution was diluted in petri dishes with a doubling dilution method. Each petri dish contained 2 ml of medical solution, and the medical solution was diluted to 20 ml, so that the final concentrations of the compound were 64, 32, 16, 8, 4, 2, 1, 0.5, 0.25, 0.125, 0.0625, 0.0313 and 0.0152 μg/mL respectively.

15 sterile 2 mL EP tubes were placed in each row. 0.9 mL of liquid medium was added to the first tube, and 0.5 mL of liquid medium was added to the other tubes respectively. 0.1 mL of prepared medical solution was added to the first tube and fully mixed. Then, 0.5 mL of medical solution was added to the second tube, and so on. Finally, 0.5 mL of medical solution was absorbed and discarded. Then, 0.5 mL of diluted bacterial solution was added to each tube; the lid was covered for blank control; the bacterial solution was incubated at 37° C.; and the growth of mycoplasma was observed.

The MIC results of target compounds against Mycoplasma gallisepticum are shown in Table 4.

TABLE 4
Results of drug sensitivity of some pleuromutilin drugs to 3
Mycoplasma gallisepticums

| | | | 352-tiamulin | |
|---|---|---|---|---|
| | | | resistant | |
| Compound | R = | 352 | strain | M47 |
| 2 | 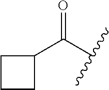 [SMILES: *C(=O)C1CCC1] | 0.008 | 0.25 | 0.5 |
| 4 | 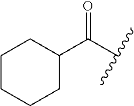 [SMILES: *C(=O)C1CCCCC1] | 0.015 | 0.25 | 1 |
| 10 | 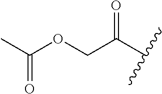 [SMILES: *C(=O)COC(C)=O] | ≤0.004 | 0.03 | 0.03 |
| 18 | 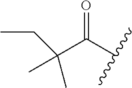 [SMILES: *C(=O)C(C)(C)CC] | 0.03 | 0.5 | 1 |
| 23 | 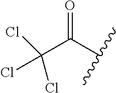 [SMILES: *C(=O)C(Cl)(Cl)Cl] | 0.125 | 1 | 4 |
| Tiamulin | | ≤0.004 | 0.25 | 0.125 |

(3) Cytotoxicity experiment

The MTT method was adopted in the experiment.

The cell strain used in the experiment was mouse mononuclear macrophage (RAW264.7).

Preparation of the target compound stock solution: 8 mg of target compound was accurately weighed, dissolved in 1 mL of DMSO, and fully shaken to obtain the stock solution (8 mg/mL); and the stock solution was filtered with a 0.22 μm filter membrane and sterilized, and stored at −20° C.

MTT incubation and OD value determination: cells in good growth state were collected, and 100 μL of cells with a density of 5×10⁵ cells/mL per well were added to a 96-well plate for plate culturing. The 96-well plate was placed in a 5% CO₂ constant temperature incubator and stood overnight. The complete culture solution was discarded; the compound to be tested with a final concentration of 4 μg/mL was added (dissolved in DMSO to prepare 4 mg/mL mother solution); and the solution was incubated in the 5% CO₂ constant temperature incubator for 24 h. The supernatant was discarded and the freshly configured MTT with 0.5 mg/mL concentration was added (dissolved in DMEM to prepare 5 mg/mL mother solution, subpackaged and stored at −20° C.) for incubation away from light for 4 h. The supernatant was discarded; and 150 μL of DMSO was added, and then the solution was shaken in a shaker at 70 r/min for 10 min at room temperature. The absorbance was measured at 490 nm by a microplate reader, and the cell survival rate was calculated. The experiment was repeated independently for three times. The formula was as follows:

$$cell survival rate ( \% ) = OD drug group / OD control group \times 100 \%$$

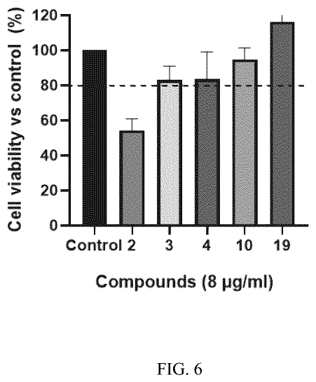

FIG. 6 shows the toxicity experiment results of some compounds. It can be seen that the target compounds are not toxic to the selected cell strain under 8 μg/mL, have certain growth promoting effects, and are suitable for use as novel antibacterial drugs to prevent and treat infectious diseases caused by the infection of human or animal or drug-resistant Staphylococcus aureus or multidrug-resistant bacteria.

The above embodiments are the preferred implementation modes of the present invention, but the implementation modes of the present invention are not limited by the above embodiments. Any other changes, modifications, substitutions, combinations and simplifications made without deviating from the spiritual essence and principle of the present invention shall be equivalent replacement modes and shall be included in the protection scope of the present invention.

We claim:

1. A pleuromutilin derivative with a thiazole side chain, which is a compound of formula 2 or a pharmaceutically acceptable salt thereof, and a solvent compound, enantiomer, diastereoisomer, tautomer, or mixture of any proportion of the compound of formula 2 or the pharmaceutically acceptable salt thereof, comprising racemic mixtures: 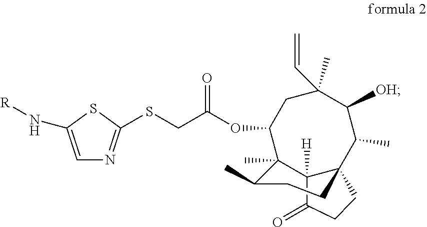
wherein R is cyclopropyl, cyclobutyl, cyclopentyl, cyclohexyl,

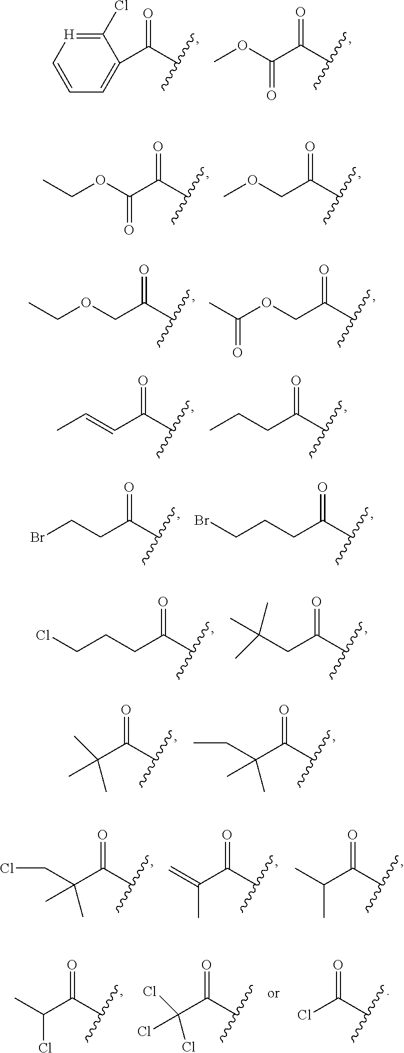

2. The pleuromutilin derivative with the thiazole side chain according to claim 1, wherein the pharmaceutically acceptable salt is a salt formed by the compound of formula 2 with hydrochloric acid, hydrobromic acid, sulfuric acid, nitric acid, phosphoric acid, acetic acid, fumaric acid, maleic acid, oxalic acid, malonic acid, succinic acid, citric acid, malic acid, methanesulfonic acid, ethanesulfonic acid, benzenesulfonic acid, toluenesulfonic acid, glutamic acid or aspartic acid.

3. The pleuromutilin derivative with the thiazole side chain according to claim 2, wherein the pharmaceutically acceptable salt has the following structural formula:

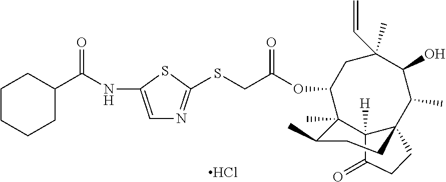 [SMILES: C=C[C@]1(C)C[C@@H](OC(=O)CSc2ncc(NC(=O)C3CCCCC3)s2)[C@]2(C)[C@H](C)CC[C@]3(CCC(=O)[C@H]32)[C@@H](C)[C@@H]1O ; Cl]

4. A preparation method for the pleuromutilin derivative with the thiazole side chain of claim 1, comprising the following steps:
(1) making pleuromutilin react with p-toluenesulfonyl chloride to obtain an intermediate I with a structure shown in formula 3;
(2) making the intermediate I prepared in step (1) react with sodium iodide to obtain an intermediate II with a structure shown in formula 4;
(3) making aminoacetonitrile hydrochloride as raw material react with CS₂ to obtain an intermediate III with a structure shown in formula 5;
(4) making the intermediate II prepared in step (2) react with the intermediate III prepared in step (3) to obtain an intermediate IV with a structure shown in formula 6;
(5) making the intermediate IV prepared in step (4) react with various acyl chlorides to obtain the pleuromutilin derivative with the thiazole side chain with a structure shown in formula 2.

5. The preparation method for the pleuromutilin derivative with the thiazole side chain according to claim 4, wherein the intermediates I, II, III and IV have structural formulas 3 to 6 respectively:

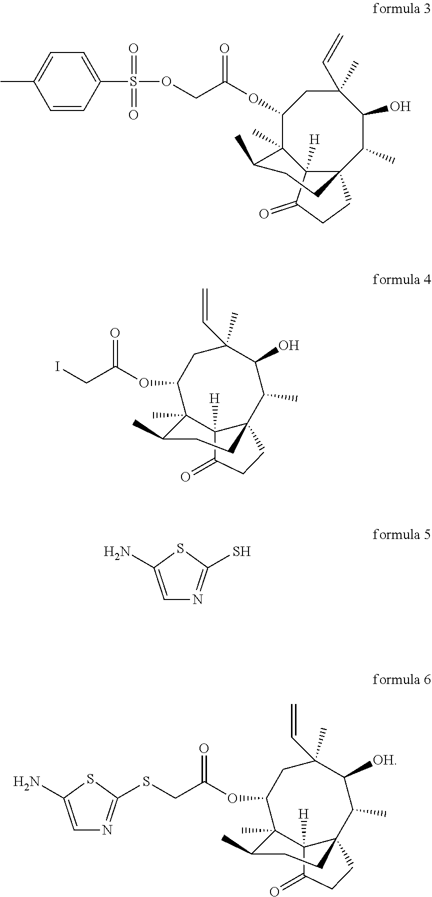 [SMILES: C=C[C@]1(C)C[C@@H](OC(=O)COS(=O)(=O)c2ccc(C)cc2)[C@]2(C)[C@H](C)CC[C@]3(CCC(=O)[C@H]32)[C@@H](C)[C@@H]1O ; C=C[C@]1(C)C[C@@H](OC(=O)CI)[C@]2(C)[C@H](C)CC[C@]3(CCC(=O)[C@H]32)[C@@H](C)[C@@H]1O ; Nc1cnc(S)s1 ; C=C[C@]1(C)C[C@@H](OC(=O)CSc2ncc(N)s2)[C@]2(C)[C@H](C)CC[C@]3(CCC(=O)[C@H]32)[C@@H](C)[C@@H]1O]

6. Application of the pleuromutilin derivative with the thiazole side chain of claim 1 in the preparation of antibacterial products.

7. The application of the pleuromutilin derivative with the thiazole side chain in the preparation of antibacterial products according to claim 6, wherein the antibacterial products are drugs for the treatment of infectious diseases.

8. The application of the pleuromutilin derivative with the thiazole side chain in the preparation of antibacterial products according to claim 7, wherein the infectious diseases are caused by infection of drug-resistant Staphylococcus aureus or multi-drug resistant bacteria in humans or animals.

9. The application of the pleuromutilin derivative with the thiazole side chain in the preparation of antibacterial products according to claim 7, wherein the drugs contain one or more pharmaceutically acceptable carriers, excipients or diluents.
